(19) United States
(12) Patent Application Publication
(10) Pub. No.: US 2019/0350543 A1
(43) Pub. Date: Nov. 21, 2019

(54) CONNECTORLESS X-RAY DETECTOR

(71) Applicant: General Electric Company, Schenectady, NY (US)

(72) Inventors: Mahesh Raman Narayanaswamy, Milwaukee, WI (US); Sean Michael Bacon, Waukesha, WI (US); Nicholas Ryan Konkle, Sussex, WI (US); Joseph John Kulak, Milwaukee, WI (US); Habib Vafi, Brookfield, WI (US)

(21) Appl. No.: 15/982,545

(22) Filed: May 17, 2018

(51) Int. Cl. A61B 6/00; H01G 11/06

(52) U.S. Cl. CPC A61B 6/4405; H01G 11/06; A61B 6/56; A61B 6/4233

(57) ABSTRACT

A digital X-ray detector is provided. The detector includes a detector array configured to generate image data based on incident X-ray radiation. The detector also includes a housing in which the detector array is disposed. The detector is configured to be inductively charged and the detector lacks any external electrical connector.

### BACKGROUND

The subject matter disclosed herein relates to X-ray imaging systems and more particularly to a connectorless portable X-ray detector.

A number of radiological imaging systems of various designs are known and are presently in use. Such systems generally are based upon generation of X-rays that are directed toward a subject of interest. The X-rays traverse the subject and impact a film or a digital detector. In medical diagnostic contexts, for example, such systems may be used to visualize internal tissues and diagnose patient ailments. In other contexts, parts, baggage, parcels, and other subjects may be imaged to assess their contents and for other purposes.

Increasingly, such X-ray systems use digital circuitry, such as solid-state detectors, for detecting the X-rays, which are attenuated, scattered or absorbed by the intervening structures of the subject. Solid-state detectors may generate electrical signals indicative of the intensities of received X-rays. These signals, in turn, may be acquired and processed to reconstruct images of the subject of interest.

As digital X-ray imaging systems have become increasingly widespread, digital X-ray detectors have become more portable for even greater versatility. Typically, these detectors include a lithium ion battery that can be recharged. However, charging times for these detectors are quite long (e.g., over 2 hours). In addition, the batteries of these detectors have a relatively short life (e.g., 2 to years). Further, bin charging of these detectors utilizes a wired connector based solution that presents compatibility issues in situations involving multiple detectors with a single imaging system. These detectors typically include a connector for detector pairing that provides a path for unwanted fluid ingress and keeps the detectors from being interchanged with other imaging systems. In addition, these detectors provide an additional path for unwanted fluid ingress via the removable battery.

### BRIEF DESCRIPTION

Certain embodiments commensurate in scope with the originally claimed subject matter are summarized below. These embodiments are not intended to limit the scope of the claimed subject matter, but rather these embodiments are intended only to provide a brief summary of possible forms of the subject matter. Indeed, the subject matter may encompass a variety of forms that may be similar to or different from the embodiments set forth below.

In accordance with a first embodiment, a digital X-ray detector is provided. The detector includes a detector array configured to generate image data based on incident X-ray radiation. The detector also includes a housing in which the detector array is disposed. The detector is configured to be inductively charged and the detector lacks any external electrical connector.

In accordance with a second embodiment, a system is provided. The system includes a charging device that includes a transmitter configured to generate a magnetic field. The system also includes a digital X-ray detector including a receiver disposed within the detector and configured to generate a current in response to the magnetic field for inductively charging the detector.

In accordance with a third embodiment, a method is provided. The method includes generating, via a transmitter, a magnetic field within a vicinity of a digital X-ray detector. The method also includes generating, via a receiver disposed within the detector, a current in response to the magnetic field. The method further includes charging a lithium-ion capacitor disposed within the digital X-ray detector. The lithium-ion capacitor is configured to power the detector.

### BRIEF DESCRIPTION OF THE DRAWINGS

These and other features, aspects, and advantages of the present invention will become better understood when the following detailed description is read with reference to the accompanying drawings in which like characters represent like parts throughout the drawings, wherein:

FIG. 1 is a diagrammatical overview of a digital X-ray imaging system in which the present technique may be utilized;

FIG. 2 is a diagrammatical overview of an embodiment of an X-ray system;

FIG. 3 is a diagrammatical overview of components of a digital X-ray detector interfacing with a charging device; and

FIG. 4 is a schematic diagram of the interaction between electrical components of the digital X-ray detector of FIG. 3.

### DETAILED DESCRIPTION

One or more specific embodiments will be described below. In an effort to provide a concise description of these embodiments, all features of an actual implementation may not be described in the specification. It should be appreciated that in the development of any such actual implementation, as in any engineering or design project, numerous implementation-specific decisions must be made to achieve the developers' specific goals, such as compliance with system-related and business-related constraints, which may vary from one implementation to another. Moreover, it should be appreciated that such a development effort might be complex and time consuming, but would nevertheless be a routine undertaking of design, fabrication, and manufacture for those of ordinary skill having the benefit of this disclosure.

When introducing elements of various embodiments of the present subject matter, the articles “a,” “an,” “the,” and “said” are intended to mean that there are one or more of the elements. The terms “comprising,” “including,” and “having” are intended to be inclusive and mean that there may be additional elements other than the listed elements. Furthermore, any numerical examples in the following discussion are intended to be non-limiting, and thus additional numerical values, ranges, and percentages are within the scope of the disclosed embodiments.

The following embodiments describe a portable digital X-ray detector that is connectorless (e.g., lacks any external electrical connector). The detector is configured to be inductively charged and includes a lithium-ion capacitor (LiC) for powering the detector. The detector may be wirelessly paired with an imaging system (e.g., QR code, Bluetooth transmission, etc.). Wirelessly pairing the detector removes the need for a physical connector for pairing. In addition, the detector may wirelessly transmit data (e.g., image data) to the imaging system and/or a remote site. In certain embodiments, the detector may compress the data to limit the transfer size for wireless transmission. In certain embodiments, an additively manufactured (e.g., three-dimensional (3D)) antenna may be utilized for wireless data transfer. The detector may be fully charged quickly (e.g., 30 minutes or less). The detector may also include a longer lifetime (e.g., 7 to 10 years) due to the LiC. Various compatibility issues will be removed with certain imaging systems due to the wireless structure of the detector. Further, due to the embedded LiC, the detector may be more rugged.

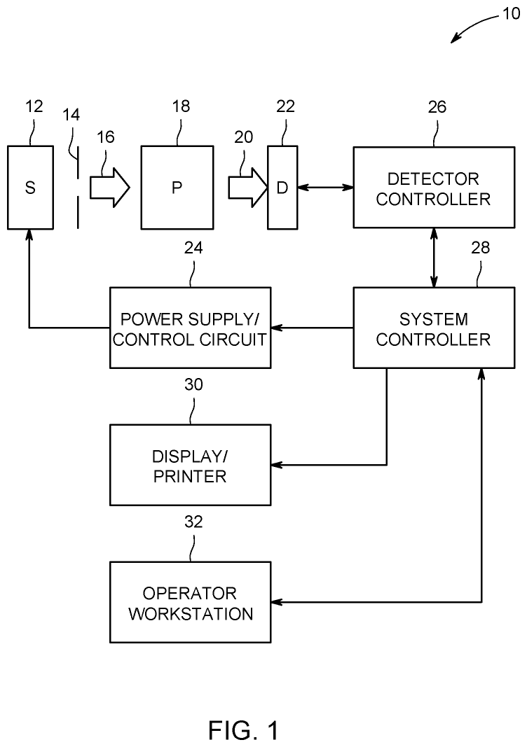

Turning now to the drawings, FIG. 1 illustrates diagrammatically an imaging system 10 for acquiring and processing discrete pixel image data. In the illustrated embodiment, system 10 is a digital X-ray system designed both to acquire original image data and to process the image data for display in accordance with the present technique. The imaging system 10 may be a stationary system disposed in a fixed X-ray imaging room or a mobile X-ray system. In the embodiment illustrated in FIG. 1, imaging system 10 includes a source of X-ray radiation 12 positioned adjacent to a collimator 14. Collimator 14 permits a stream of radiation 16 to pass into a region in which a subject, such as a human patient 18 is positioned. A portion of the radiation 20 passes through or around the subject and impacts a digital X-ray detector, represented generally at reference numeral 22. The detector 22 is portable. In certain embodiments, the detector 22 may convert the X-ray photons incident on its surface to lower energy photons, and subsequently to electric signals, which are acquired and processed to reconstruct an image of the features within the subject. In other embodiments, such as in a direct conversion implementation, the incident radiation itself may be measured without an intermediary conversion process.

Source 12 is controlled by a power supply/control circuit 24 which furnishes both power and control signals for examination sequences. Moreover, detector 22 is coupled to a detector controller 26 which commands acquisition of the signals generated in the detector 22. Detector controller 26 may also execute various signal processing and filtration functions, such as for initial adjustment of dynamic ranges, interleaving of digital image data, and so forth. Both power supply/control circuit 24 and detector controller 26 are responsive to signals from a system controller 28. In general, system controller 28 commands operation of the imaging system to execute examination protocols and to process acquired image data. In the present context, system controller 28 also includes signal processing circuitry, typically based upon a general purpose or application-specific digital computer; and associated manufactures, such as optical memory devices, magnetic memory devices, or solid-state memory devices, for storing programs and routines executed by a processor of the computer to carry out various functionalities (e.g., gain calibration and gain correction), as well as for storing configuration parameters and image data; interface protocols; and so forth. In one embodiment, a general or special purpose computer system may be provided with hardware, circuitry, firmware, and/or software for performing the functions attributed to one or more of the power supply/control circuit 24, the detector controller 26, and/or the system controller 28 as discussed herein.

In the embodiment illustrated in FIG. 1, system controller 28 is linked to at least one output device, such as a display or printer as indicated at reference numeral 30. The output device may include standard or special purpose computer monitors and associated processing circuitry. One or more operator workstations 32 may be further linked in the system for outputting system parameters, requesting examinations, viewing images, and so forth. In general, displays, printers, workstations, and similar devices supplied within the system may be local to the data acquisition components, or may be remote from these components, such as elsewhere within an institution or hospital, or in an entirely different location, linked to the image acquisition system via one or more configurable networks, such as the Internet, virtual private networks, and so forth.

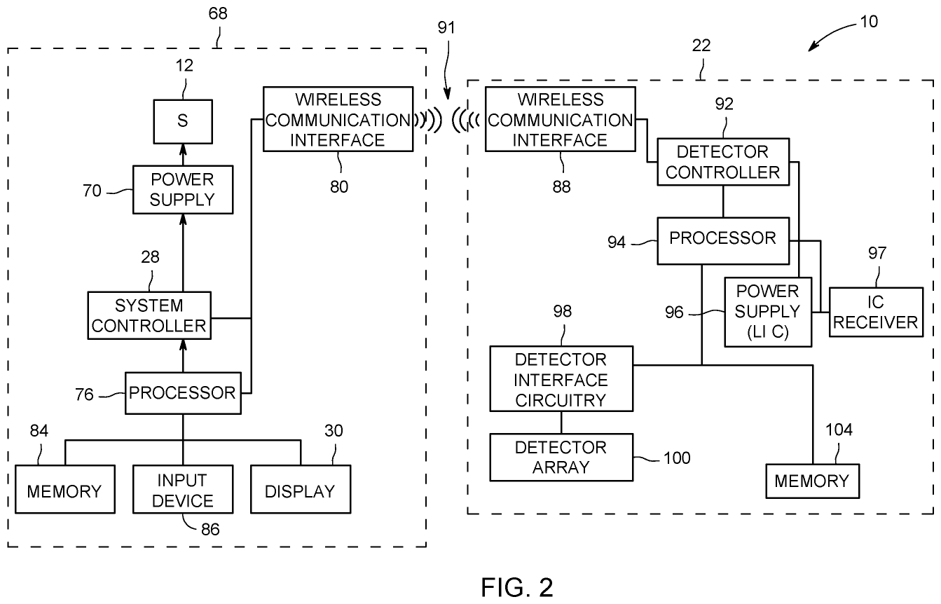

FIG. 2 illustrates diagrammatically the imaging system 10 of FIG. 1. An imager system 68 includes the X-ray source 12 of radiation. The X-ray source 12 is controlled by a power supply 70, which furnishes both power and control signals for examination sequences. The power supply 70 is responsive to signals from a system controller 28. In general, the system controller 28 commands operation of the imaging system to execute examination protocols and to process acquired image data. In the present context, the system controller 28 also includes signal processing circuitry, typically based upon a general purpose or application-specific digital computer, associated memory circuitry for storing programs and routines executed by the computer, as well as configuration parameters and image data, interface circuits, and so forth. The system controller 28 may include or may be responsive to a processor 76. The processor 76 receives image data from the detector 22 and processes the data to reconstruct an image of a subject.

The processor 76 is linked to a wireless communication interface 80 that allows wireless communication with the detector 22. In certain embodiments, the wireless communication interface 80 also enables wireless pairing with the detector 22. The imager system 12 may also be in communication with a server. The processor 76 is also linked to a memory 84, an input device 86, and the display 30. The memory 84 stores configuration parameters, calibration files received from the detector 22, and lookup tables used for image data processing. The input device 86 may include a mouse, keyboard, or any other device for receiving user input, as well as to acquire images using the imager system 12. The display 30 allows visualization of output system parameters, images, and so forth.

The detector 22 includes a wireless communication interface 88 (e.g., antenna) for wireless communication with the imager system 12. In certain embodiments, the interface 88 may be additively manufactured (e.g., 3D printed) on an external surface of the detector 22 (e.g., detector cover) or within the detector 22 (e.g., on the imager). In certain embodiments, the wireless communication interface 80 also enables wireless pairing with the imager system 12. The detector 22 may also be in communication with a server. The wireless communication interfaces 80 and 88 define a communication channel 91 between the imager system 12 and the detector 22, over which digital X-ray images are transmitted. It is noted that the wireless communication interface 88 may utilize any suitable wireless communication protocol, such as an ultra-wideband (UWB) communication standard, a Bluetooth communication standard, or any 802.11 communication standard. Wirelessly pairing between the imager system 12 and the detector 22 may occur over these interfaces 80, 88 (e.g., utilizing Bluetooth communication standard or another communication standard). In certain embodiments, the imager system 12 and the detector 22 may be wirelessly paired utilizing QC codes or any other method of wirelessly pairing. Wireless pairing eliminates the presence of any fluid ingress path for any biological fluids to enter the detector 22 due to the absence of any connector based system for pairing. Moreover, the detector 22 is coupled to a detector controller 92 which coordinates the control of the various detector functions. For example, the detector controller 92 may execute various signal processing and filtration functions, such as for initial adjustment of dynamic ranges, interleaving of digital image data, and so forth. The detector controller 92 is responsive to signals from the system controller 28, as well as the detection circuitry 78. The detector controller 92 is linked to a processor 94. The processor 94, the detector controller 92, and all of the circuitry receive power from a power supply 96. The power supply 96 is disposed (e.g., embedded) within the housing of the detector 22. This eliminates the presence of any fluid ingress path for any biological fluids to enter the detector 22. The power supply 96 includes a supercapacitor (e.g., one or more LiC). The power supply 96 is configured to be inductively charged. The power supply 96 and the processor 94 are coupled to an inductive charge receiver 97 (e.g., receiver coil). The inductive charge receiver 97 is configured to generate a current (e.g., AC current) in response to a magnetic field transmitted by a transmitter coil of a charging device (e.g., charging pad). The current generated by the inductive charge receiver 97 is converted into a direct current for charging the power supply 96.

Also, the processor 94 is linked to detector interface circuitry 98. The detector 22 converts X-ray photons received on its surface to lower energy photons. The detector 22 includes a detector array 100 that includes an array of photodetectors to convert the light photons to electrical signals. Alternatively, the detector 22 may convert the X-ray photons directly to electrical signals. These electrical signals are converted to digital values by the detector interface circuitry 98 which provides the values to the processor 94 to be converted to imaging data and sent to the imager system 12 to reconstruct an image of the features within a subject. Alternatively, the imaging data may be sent from the detector 22 to a server to process the imaging data. The processor 94 may compress the image data sent over the communication channel 91. Further, the processor 94 is linked to a memory 104. The memory 104 may store various configuration parameters, calibration files, and detector identification data.

The detector 22 lacks any external electrical connector on the housing due to the embedded power supply 96 that can be wirelessly charged and the ability of the detector 22 to wirelessly pair with the imaging system 10 and to wirelessly communicate data to the system 10. In addition, due to these aspects, the detector 22 also lacks fluid ingress paths for biological fluid to enter the detector 22.

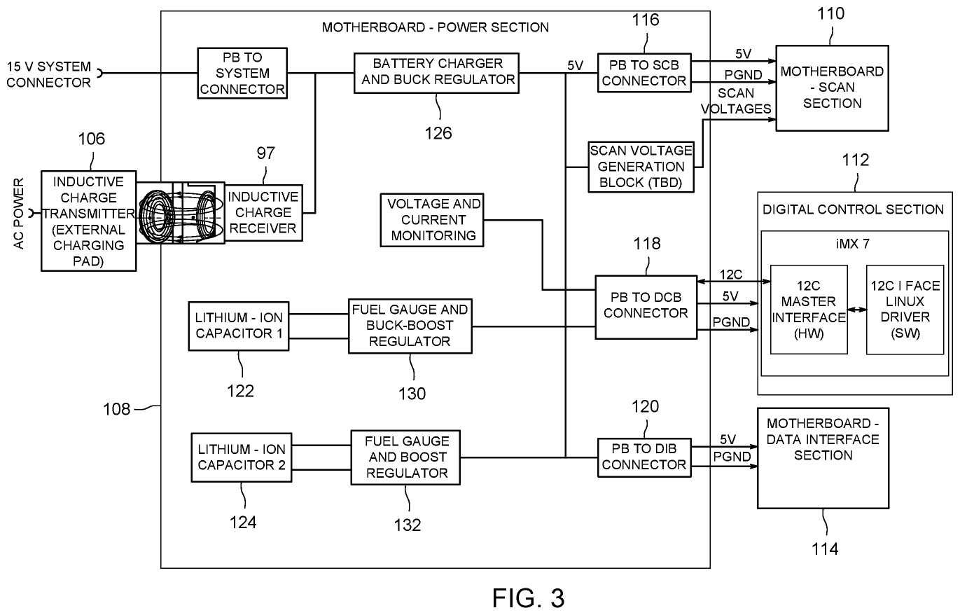

FIG. 3 is a diagrammatical overview of components of the digital X-ray detector 22 interfacing with a charging device 106. The detector 22 includes a power section 108 of a motherboard, a scan section 110 of the motherboard, a digital control section 112, and a data interface section 114 of the motherboard. The power section 108 is coupled to the sections 110, 112, and 114 via connectors 116, 118, and 120. The scan section 110 regulates which columns of pixels are selected, while the data control section 114 reads any signals (e.g., signals generated responsive to impingement of light upon a pixel) present across one or more rows of pixels. Together, the scan section 110 and the data control section 114 collect and organize the signals from the detector 22 for use in reconstructing an image. The digital control section 112 acts as the master that regulates interaction between the detector 22 (as well as different components of the detector 22) and the imaging system 10.

The power section 108 regulates the power supply of the detector 22. The power section 108 also regulates the inductive charging of the power supply. The main components of the power section 108 include the inductive charge receiver 97 and the LiCs 122, 124 (which form the power supply). The number of LiCs that form the power supply of the detector 22 may vary. As noted above, power section 108 includes the inductive charge receiver 97 (e.g., coil) for inductive charging of the LiCs 122, 124. As depicted, the charging device 106 (e.g., inductive charge transmitter) includes a coil for emitting a magnetic field in response to an AC current provided to the coil via transmitter circuitry. One or more charging devices 106 may be part of the imaging system 10 (e.g., coupled to or stored within a component (e.g., bin) of the system 10 or imager system 68) or distributed throughout a medical or imaging facility for charging the detector 22. In response to the magnetic field transmitted by the transmitter coil of the charging device 106 (e.g., charging pad), the receiver 97 is configured to generate a current (e.g., AC current). The current generated by the inductive charge receiver 97 is converted into a direct current (e.g., via receiver circuitry) that is utilized in charging the LiCs 122, 124. In certain embodiments, the power section 108 may include other components. For example, the power section 108 may include a buck charger (e.g., NMOS-NMOS synchronous buck converter) and buck regulator (e.g., step-down converter) 126 for charging the LiCs 122, 124. In addition, the power section 108 may include a fuel gauge and a buck-boost regulator 130 (e.g., for step-up/step-down DC/DC conversion) coupled to LiC 122, and a fuel gauge and boost regulator 132 coupled to LiC 124.

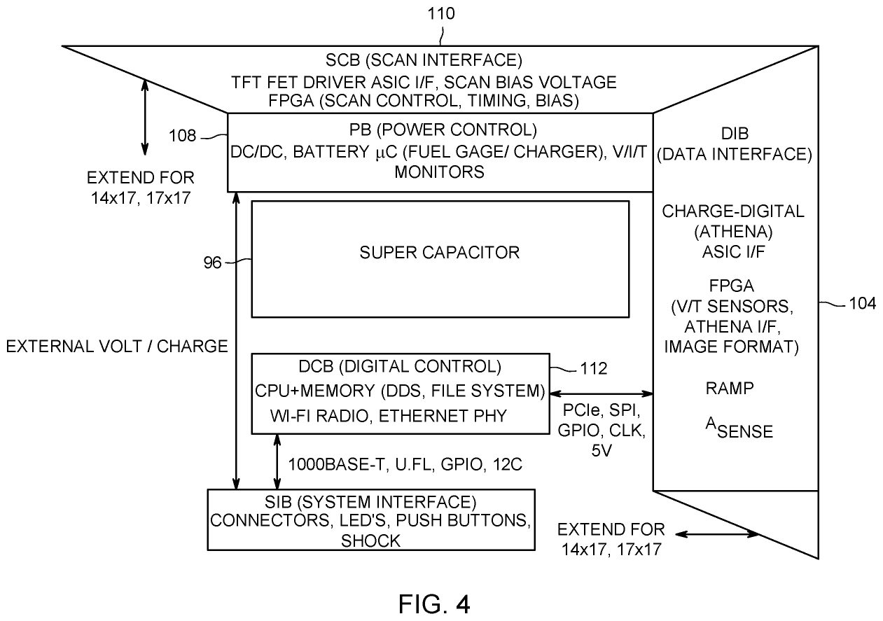

The power supply of the detector 22 is configured to be inductively charged in 30 minutes or less (at a standard inductive wireless charging power of 15 watts that results in charge current of approximately 2-3 amperes). The life of the LiCs 122, 124 may range from 7 to 10 years. Although the power supply is described above as a LiC, the power supply may be any other supercapacitor or device capable of being rapidly charged (in 30 minutes or less). For example, the power supply could be one or more lithium-titanate oxide (LTO) batteries. The interaction between the electrical components of the detector 22 described above are further illustrated in FIG. 4.

Technical effects of the disclosed embodiments include providing a digital X-ray detector lacking any external connectors (e.g., electrical connectors) that can be wirelessly charged (e.g., inductively charged) rather quickly (e.g., 30 minutes or less). The digital X-ray detector may include a supercapacitor (e.g., LiC) embedded within the detector to increase the ruggedness of the detector. The supercapacitor may provide a longer life energy storage device for the detector, thus, providing a higher overall system reliability. In addition, the detector may wirelessly pair with the imaging system to avoid compatibility issues with different imaging systems.

This written description uses examples to disclose the invention, including the best mode, and also to enable any person skilled in the art to practice the invention, including making and using any devices or systems and performing any incorporated methods. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal languages of the claims.

1. A digital X-ray detector, comprising:
a detector array configured to generate image data based on incident X-ray radiation; and
a housing in which the detector array is disposed, wherein the digital X-ray detector is configured to be inductively charged and the digital X-ray detector lacks any external electrical connector, and wherein the digital X-ray detector lacks any fluid ingress paths.

2. The digital X-ray detector of claim 1, comprising a lithium-ion capacitor device disposed within the housing and configured to be inductively charged and to power the digital X-ray detector.

3. The digital X-ray detector of claim 1, comprising a receiver disposed within the housing and configured to generate a current in response to a magnetic field generated by a transmitter in a charging device.

4. The digital X-ray detector of claim 1, wherein the digital X-ray detector is configured to be wirelessly paired with an imaging system.

5. The digital X-ray detector of claim 1, wherein the digital X-ray detector is configured to wirelessly transmit data to an imaging system via an antenna.

6. The digital X-ray detector of claim 5, wherein the antenna is disposed directly on the housing via additive manufacturing.

7. The digital X-ray detector of claim 5, wherein the antenna is disposed within the digital X-ray detector via additive manufacturing.

8. (canceled)

9. A system, comprising:
a charging device comprising a transmitter configured to generate a magnetic field; and
a digital X-ray detector comprising a receiver disposed within the digital X-ray detector and configured to generate a current in response to the magnetic field for inductively charging the digital X-ray detector, wherein the digital X-ray detector is configured to wirelessly transmit data to an imaging system via an antenna, and the antenna is disposed directly on a housing of the digital X-ray detector or within the digital X-ray detector via additive manufacturing.

10. The system of claim 9, wherein the digital X-ray detector comprises a lithium-ion capacitor device disposed within the digital X-ray detector that is configured to be inductively charged.

11. The system of claim 9, wherein the digital X-ray detector is configured to be fully charged via inductive charging in 30 minutes or less.

12. The system of claim 9, wherein the imaging system comprises an X-ray source, and wherein the imaging system and the digital X-ray detector are configured to wirelessly pair with each other.

13.-15. (canceled)

16. The system of claim 9, wherein the digital X-ray detector lacks any external electrical connector.

17. A method, comprising:
generating, via a transmitter, a magnetic field within a vicinity of a digital X-ray detector;
generating, via a receiver disposed within the digital X-ray detector, a current in response to the magnetic field;
charging a lithium-ion capacitor disposed within the digital X-ray detector, wherein the lithium-ion capacitor is configured to power the digital X-ray detector, and wherein the digital X-ray detector lacks any external electrical connector, wherein the digital X-ray detector lacks any fluid ingress paths.

18. The method of claim 17, comprising wirelessly transmitting, via an antenna of the digital X-ray detector, data to an imaging system.

19. The method of claim 17, comprising wirelessly pairing the digital X-ray detector to an imaging system.

20. (canceled)

21. The system of claim 9, wherein the digital X-ray detector lacks any fluid ingress paths.

22. (canceled)
